(19) United States
(12) Patent Application Publication
(10) Pub. No.: US 2021/0330918 A1
(43) Pub. Date: Oct. 28, 2021

(54) INTUBATION CHAMBER

(71) Applicants: Michael N. Menezes, Conroe, TX (US); Charles Scott Rippetoe, The Woodlands, TX (US)

(72) Inventors: Michael N. Menezes, Conroe, TX (US); Charles Scott Rippetoe, The Woodlands, TX (US)

(21) Appl. No.: 16/859,343

(22) Filed: Apr. 27, 2020

(51) Int. Cl. A61M 16/04; A61M 16/00

(52) U.S. Cl. CPC A61M 16/0472; A61M 16/0463; A61M 16/0488; A61M 16/0051; A61M 16/0003; A61M 16/0057

(57) ABSTRACT

An intubation chamber comprising a vertical member that comprises at least one arm port, an enclosure member comprising at least one access port; and a transparent angled member coupled to the vertical member and the enclosure member, said at least one arm port to allow access to intubate a patient.

### TECHNICAL FIELD

This disclosure generally relates to the field of medicine, and, in particular, to an intubation chamber to prevent the contamination of medical personnel.

### BACKGROUND

The claims of this patent, when issued, will be licensed by the inventors and Texas Torque for a nominal royalty payable to Texas Torque. Texas Torque is an organization dedicated to encourage high school students to pursue technical careers. Infectious diseases are commonly spread through direct contact between an infected person and a healthy person through handshaking or kissing, or by indirect contact such as breathing, coughing or sneezing wherein particulates from the infected person are transferred to the healthy person. In an ambulance transferring an infected person to a hospital, or in a hospital, medical procedures such intubations are performed to aid a person who is in respiratory distress to get air into their lungs.

Intubation is a process of inserting a tube, called an endotracheal tube through either the infected person's or patient's mouth or through a cut in the front of the neck into that patient's trachea and securing the tube in place. This is done so that the patient who is in respiratory distress can be placed on a ventilator to assist that patient with breathing. The terms tracheostomy or trach may be used to refer to both the surgical procedure and to the opening created by the procedure.

Often times during the intubation process the patient coughs or sneezes, or even the act of breathing may cause bodily fluids or particulates to be propelled into the air during the insertion of the tube into the patient's trachea dispersing infectious particulates into the atmosphere which can put the ambulance personnel or the doctors performing the intubation at risk of contracting the patient's infectious disease.

Conventional chambers used to prevent the spread of infection during patient intubation are typically a box type device made of polycarbonate or other transparent material, that is placed over a patient with arm ports for a doctor or medical technician to perform an intubation procedure. The disadvantage of this box type design is that a doctor or medical technician is unable to get close enough to the patient to observe the interaction of the laryngoscope or other instruments with the tongue, epiglottis, larynx, vocal cords, trachea and airways of the patient in order to easily perform the intubation process.

### BRIEF DESCRIPTION OF THE DRAWINGS

Embodiments of the disclosure may be better understood by referencing the accompanying drawings

FIG. 1 is a perspective view of an example intubation chamber, according to some embodiments.

FIG. 2 is a right side view of the intubation chamber of FIG. 1, according to some embodiments.

FIG. 3 is a left side view of the intubation chamber of FIG. 1, according to some embodiments.

FIG. 4 is a front view of the intubation chamber of FIG. 1, according to some embodiments.

FIG. 5 is a back view of the intubation chamber of FIG. 1, according to some embodiments.

FIG. 6 is a perspective view of another example intubation chamber, according to some embodiments.

FIG. 7 is an example of a coupler used to connect various parts of the intubation chamber, according to some embodiments.

FIG. 8 is a flowchart describing a method to use the intubation chamber, according to some embodiments.

### DESCRIPTION OF EMBODIMENTS

The description that follows includes example systems, methods, and techniques that embody embodiments of the disclosure. However, it is understood that this disclosure may be practiced without these specific details. For instance, this disclosure refers to the use of an intubation chamber to prevent the spread of infectious diseases from an infected patient to medical personnel using angled sides and/or in illustrative examples. However, depending on the manufacturing procedures embodiments of this disclosure can also be used wherein the edges are curved using for example blow molding. In other instances, well-known manufacturing processes, protocols, structures and techniques have not been shown in detail in order not to obfuscate the description.

While the aspects of the disclosure are described with reference to various implementations and exploitations, it will be understood that these aspects are illustrative and that the scope of the claims is not limited to them. Many variations, modifications, additions, and improvements are possible.

Example embodiments include a device that can be placed over a patient so that the infectious particulates expelled by the patient during the tracheostomy or intubation process are contained and not transmitted to ambulance personnel or the doctors performing an intubation or tracheostomy.

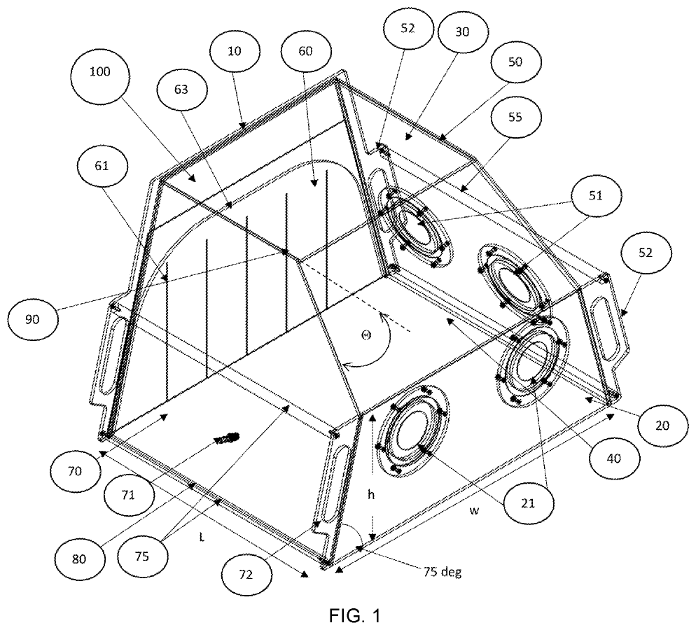

Referring to the drawings, FIG. 1 shows a perspective view of an intubation chamber 10, according to some embodiments. The intubation chamber 10 comprises a vertical member 20 comprising arm ports 21. The intubation chamber 10 further comprises enclosure member 30 with one or more access ports 51. Although the access ports 51 are shown on the right side of intubation chamber 10, the left and right sides can be interchangeable and the access ports 51 could be on the left side and the nozzle 71 could be on the right side of the intubation chamber. Also, enclosure member 30 may comprise discrete parts such as upper member 90, back member 100, left side wall 70, right side wall 50, and vertical member 20 or an integrated enclosure member that combines one or more of the discrete parts 90, 100, 70, 50 and 20. In some embodiments, arm ports 21 and access ports 51 can be sealable. Coupled to the vertical member 20 and the enclosure member 30 is a transparent angled member 40. In some embodiments, the transparent angled member 40 may be made of polycarbonate, plastic, or polymethyl methacrylate. In some embodiments, the intubation chamber 10 is transparent and may be made of polycarbonate, plastic or polymethyl methacrylate. The purpose for the transparency and angular shape of the intubation chamber 10 and in particular, for the transparent angled member 40, is for medical personnel to get a closer and clearer view of a patent within the intubation chamber 10, as compared to conventional box type intubation chambers, and to perform the intubation without the patient infecting the medical personnel performing the intubation procedure.

In some embodiments, intubation chamber 10, and in particular enclosure member 30 may comprise a back member 100 comprising a sealable opening 60 with a curtain 61. Back member 100 may be coupled to upper member 90. In some embodiments, the angle between back member 100 and upper member 90 is ninety (90) degrees. In other embodiments the angle between back member 100 and upper member 90 may be greater than 90 degrees. The sealable opening 60 seals against the torso of the patient that is being intubated using gasket 63 or other sealable means such as an inflatable seal, curtains and the like. Curtain 61 can be composed of plastic, preferable a transparent plastic, though other sealable materials may also be used. Enclosure member 30 may further comprise a left side wall 70. Left side wall 70 may comprise a nozzle 71, preferable with a back flow preventable valve, that is used to couple to a suction pump, not shown, to maintain a negative pressure within the intubation chamber 10. This negative pressure creates a vacuum within intubation chamber 10 to suck out of the intubation chamber 10 any infectious particulates that may contaminate the medical personnel performing the intubation procedure.

In some embodiments of the intubation chamber 10, the enclosure member 30 further comprises a right side wall 50. In some embodiments, the left side wall 70 and the right side wall 50 are inwardly bent at an angle of less than 90 degrees and preferably 85 degrees with the base of intubation chamber 10 as shown in FIG. 1.

In some embodiments, the bottom end of the intubation chamber 10 comprises a bottom end gasket 80 to seal the intubation chamber 10 to a gurney or table, not shown. In particular, the bottom end of intubation chamber 10, i.e., the left sidewall 70, right side wall 50 and vertical member 20, will have a gasket to seal with the gurney or table. In some embodiments, gasket 63 of sealable opening 60 of back member 100 of the intubation chamber 10, seals against the body of a patient that is being intubated using an inflatable seal. The bottom end of the intubation chamber 10 is open so that the chamber can be lifted and placed onto a patient that is lying on a table or gurney. The material used for gasket 63 and gasket 80 may be elastomeric materials or other well-known sealable materials. In some embodiments, the seal between intubation chamber 10 and the patient may be accomplished by coupling one or more straps, not shown, from the intubation chamber 10 to the table. In other embodiments, the strap may be passed from the intubation chamber 10, under a patient's body, or under the table on which the patient is laying and coupling the strap to the other side of intubation chamber 10 so that the gasket 63 fits snugly to the torso of the patient being intubated. Thus, the bottom end of gasket 80 coupled with gasket 63 of the intubation chamber 10, and/or curtain 61 forms a seal around the patient so that any infectious airborne particulates are suctioned out through nozzle 71 of intubation chamber 10, thereby keeping the medical personnel performing the intubation from being infected.

In some embodiments, the vertical member 20 has dimensions of approximately ‘h’=11.5 inches height. In some embodiments, the minimum height of the vertical member 20 is limited by the diameter of arm ports 21. In some embodiments, the arm port 21 is at least 3 inches in diameter. In some embodiments, the width ‘w’ of the intubation chamber 10 is limited by the width of the gurney or table on which the patient lays during the intubation procedure. In some embodiments, the width ‘w’ is 30 inches. In some embodiments, the length 1′ of the intubation chamber 10 is 22.5 inches.

The enclosure member 30 can include an upper member 90 coupled to back member 100 and to transparent angled member 40. In some embodiments, the angle Theta Θ between the upper member 90 and the transparent angled member 40, as shown in FIG. 1, is greater than 5 degrees, but is preferably greater than 18 degrees. In the case of an integrated enclosure member 30 the angle Theta will be measured between a plane comprising the uppermost part of upper member 90 and a plane of transparent angled member 40.

In some embodiments, the left side wall 70, and the right side wall 50 of enclosure member 30 has inwardly sloping walls. As shown in FIG. 1, the inwardly sloping side walls can have an angle of 85 degrees with the base of vertical member 20.

In some embodiments, the left side of vertical member 20 has a handle 72, and/or one or more bar handles 75 coupled thereon. Correspondingly, the right side of vertical member 20 may have a handle 52, and/or one or more bar handles 55 coupled thereon. In some embodiments, the bar handles couple the vertical member 20 to the back member 100 to enable a single individual to lift and move the intubation chamber 10 as necessary.

In some embodiments, the enclosure member 30 is coupled to vertical member 20 and transparent angled member 40 using methylene chloride based acrylic cement or plastic solvent. In other embodiments, the blow molding technique may be used to form the integrated enclosure member 30 comprising one or more of the discrete parts that includes upper member 90, back member 100, left side wall 70, right side wall 50, vertical member 20, and transparent angled member 40. In some embodiments, vertical member 20 and back member 100 may be inwardly sloped for example, to ensure stacking of multiple units for storage or shipping. In some embodiments, transparent angled member 40 may be coupled with the integrated enclosure member 30 using means well known in the art for example methylene chloride based acrylic cement or plastic solvent, hook and slots, press fitting, etc. In some embodiments, integrated enclosure member 30 may include angled member 40 so long as the angle Theta as described above is maintained. In some embodiments, the transparent angled member 40 and upper member 90 may have tabs and slots that are alternately spaced between the transparent angled member 40 and upper member 90 to prevent bowing or sagging as is well known in the art. Tabs and slots or other stiffening means may be employed between other members as well to prevent bowing or sagging.

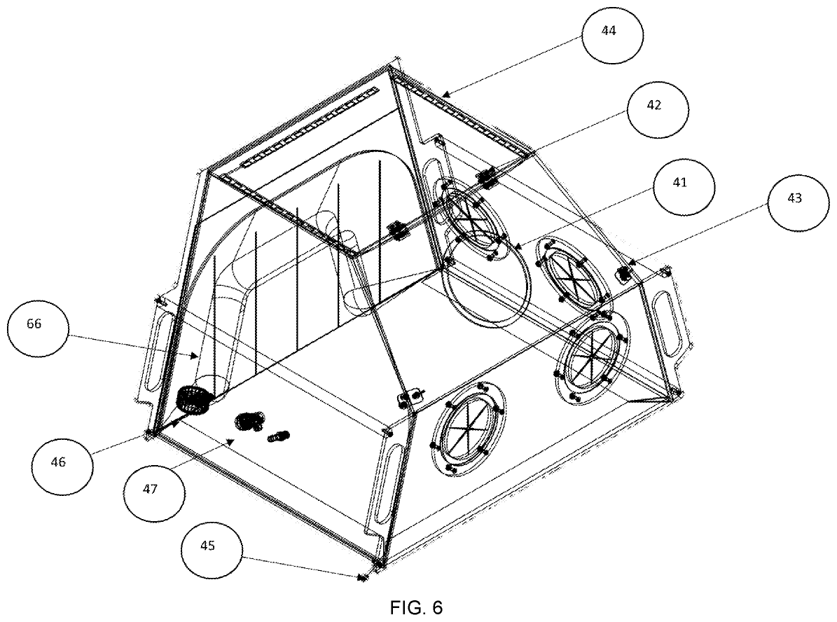

In some embodiments, the transparent angled member comprises a lens for example a convex lens, or a Fresnel lens see FIG. 6 to enable the medical personnel performing the intubation to better view the tongue, epiglottis, larynx, vocal cords, trachea and airways of the patient to perform the intubation process.

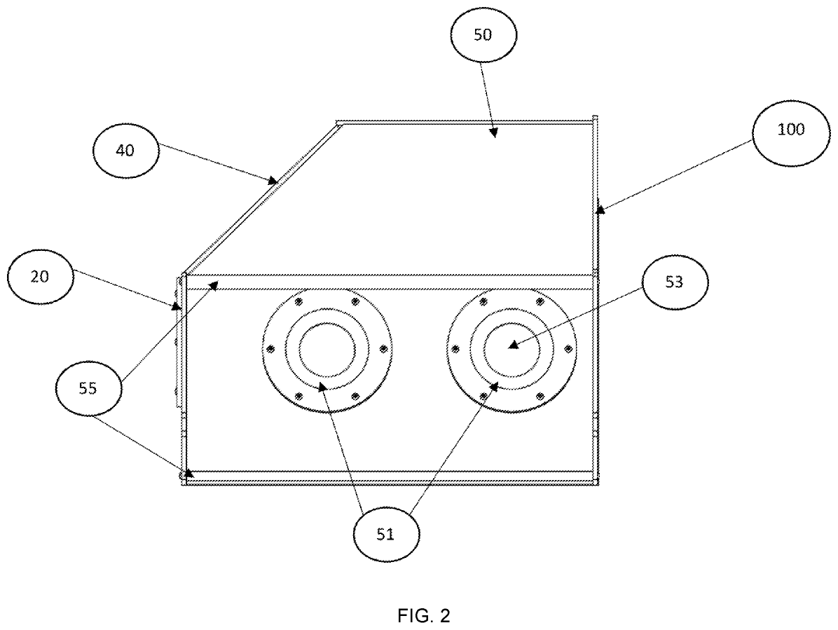

FIG. 2 is a right side view of intubation chamber of FIG. 1, according to some embodiments. As seen in FIG. 2 the right side view of intubation chamber 10 comprises a right side wall 50 and one or more access ports 51. In some embodiments, the access ports 51 are sealable using slits in the composite, elastomers, or rubber forming a seal over the arms of a user of access ports 51, and may comprise iris ports 53. Iris ports are well known in the art and can protect the hands of the medical personnel from becoming infected by any infected material expelled into the air by the infected patient within the intubation chamber 10. Also, seen in FIG. 2 are bar handles 55 that couple the vertical member 20 to back member 100, and transparent angled member 40 coupled to vertical member 20.

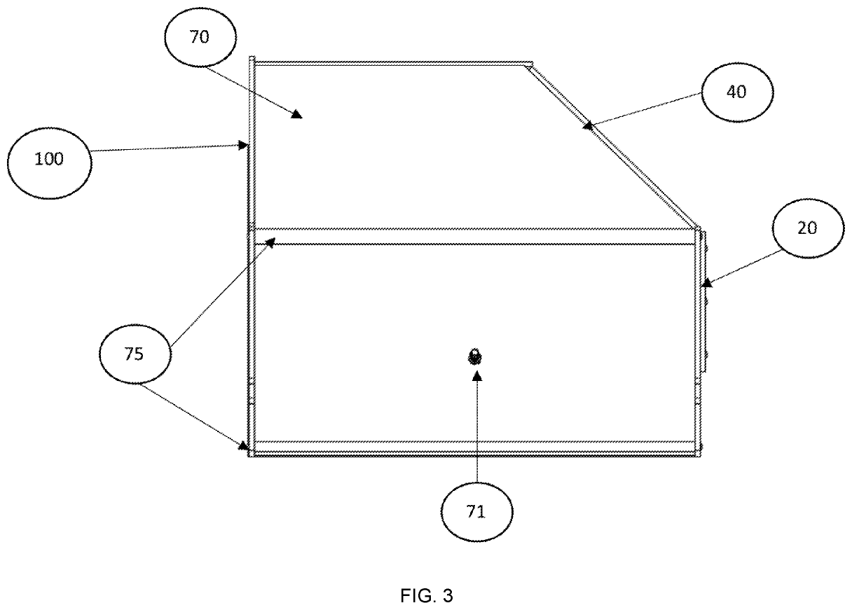

FIG. 3 is a left side view of intubation chamber of FIG. 1, according to some embodiments. As seen in FIG. 3 the left side view of intubation chamber 10 comprises enclosure member 30 that comprises a left side wall 70 and a nozzle 71. The nozzle 71 is used to couple to a suction pump, not shown, to maintain a negative pressure within the intubation chamber 10. This negative pressure is in effect a vacuum to remove from the intubation chamber 10 infectious particulates that may contaminate the medical personnel performing the intubation procedure. The nozzle 71 may be affixed with a back flow prevention valve to shut off the supply of any air that may inadvertently flow from the suction pump back into the intubation chamber 10. Also seen in FIG. 3 are bar handles 75 that couple the vertical member 20 to back member 100, and transparent angled member 40 coupled to vertical member 20.

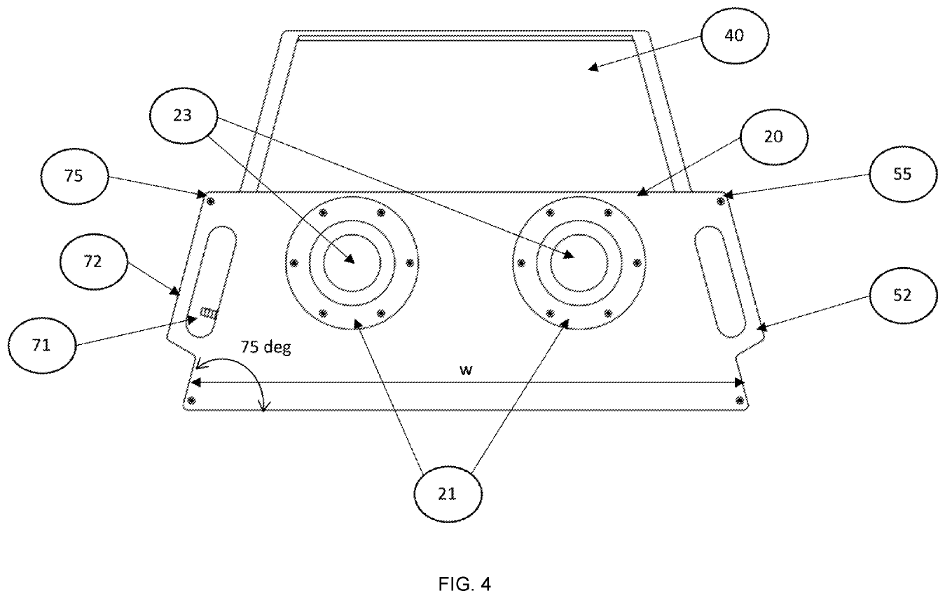

FIG. 4 is a front view of the intubation chamber of FIG. 1, according to some embodiments. As disclosed in FIG. 4, the intubation chamber 10 includes a vertical member 20 and a transparent angled member 40. The vertical member 20 comprises one or more arm ports 21. In some embodiments, the arm ports 21 are sealable, using slits in the composite, elastomers, or rubber forming a seal over the arms of a user of access ports 21. In some embodiments, the vertical member 20 has dimensions of approximately ‘h’=11.5 inches height. The minimum height of the vertical member 20 can be limited by the diameter of sealable arm port 21. In some embodiments, the arm port 21 is at least three inches in diameter. The width ‘w’ of the intubation chamber 10 can be limited by the width of the gurney or table on which the patient lays during the intubation procedure. In some embodiments, the width ‘w’ is 30 inches. In some embodiments, the arm ports comprise iris ports 23. Iris ports are well known in the art and protect the hands of the medical personnel from becoming infected by any infected material expelled into the air by the infected patient within the intubation chamber 10. In some embodiments, the left and right sides of vertical member 20 are inwardly sloped and make an angle of approximately 85 degrees with the base of vertical member 20. Also shown in FIG. 4 is nozzle 71.

Vertical member 20 can include handles 72 and 52 on the left and right side respectively of vertical member 20 as shown. Bar handles 75 and 55 extend backwardly from vertical member 20 toward the back member 100, not shown, and make it possible for a single individual to lift and move intubation chamber 10.

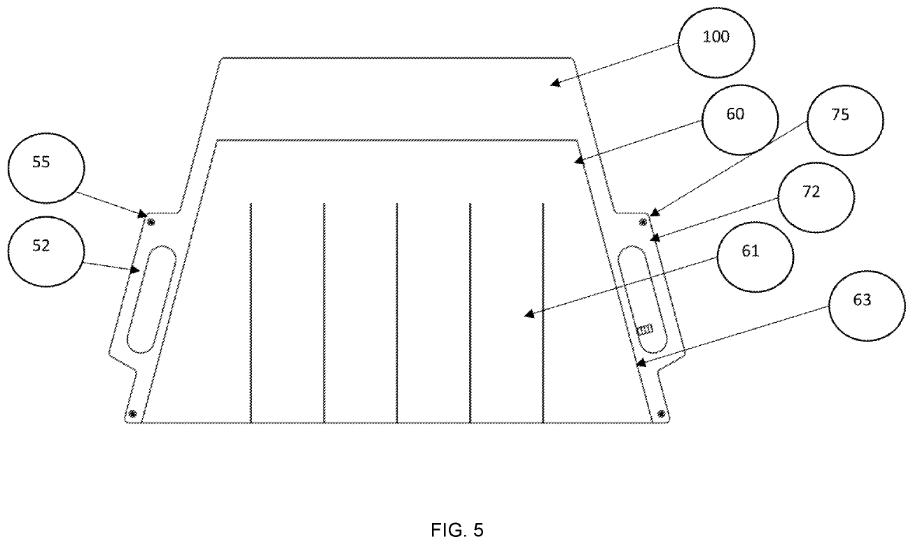

FIG. 5 is a back view of the intubation chamber of FIG. 1, according to some embodiments. As described with reference to FIG. 1, intubation chamber 10 further comprises a back member 100 comprising a sealable opening 60 with a curtain 61. Back member 100 is coupled to upper member 90, not shown. In some embodiments, the angle between back member 100 and upper member 90 is ninety (90) degrees. In other embodiments the angle between back member 100 and upper member 90 may be greater than 90 degrees. The sealable opening 60 seals against the torso of the patient that is being intubated using gasket 63. In some embodiments, back member 100 comprises handles 72 and 52 on the right and left side of back member 100 as shown. Bar handles 75 and 55 extend from back member 100 toward the vertical member 20 and make it possible for a single individual to lift and move intubation chamber 10.

FIG. 6 is a perspective view of the intubation chamber of FIG. 1, according to some embodiments. The elements of FIG. 1 are incorporated by reference into the embodiment of FIG. 6. However, in some instances the differences between FIG. 1 and FIG. 6 will be discussed in this paragraph. One skilled in the art will appreciate that one or more of the differences shown in FIG. 6 may be used in the embodiment of FIG. 1. In some embodiments, the transparent angled member 40 comprises a lens 41 for example a convex lens, or a Fresnel lens to enable the medical personnel performing the intubation to better view the tongue, epiglottis, larynx, vocal cords, trachea and airways of the patient to perform the intubation process. The transparent angled member 40 can include one or more hinges 42 and locking mechanism 43 to access the interior of intubation chamber 10. The interior of intubation chamber 10 can have one or more lights 44 to illuminate the patient during the intubation process. The lights may be battery operated or may be connected to an external power source via grommets, not shown, and connector 45. In some embodiments, the gasket 63 of sealable opening 60 of the intubation chamber 10 seals against the body of a patient that is being intubated using a material comprising elastomers, well known in the art. In some embodiments, the gasket or seal comprises an inflatable seal 66. The inflatable seal 66 of FIG. 6 may comprise elastomers or other suitable material well known in the art to form a seal against the torso of the patient. One skilled in the art will appreciate the inflatable seal 66 may be inflated via a nozzle using for example, a hand pump or a Carbon Dioxide or compressed air cartridge. In some embodiments the inflatable seal 66 and/or curtain 61 may be removable and disposable after using the intubation chamber 10 to intubate a patient. The intubation chamber 10 can include a pressure sensor 46 coupled to alarm 47 that sounds if the pressure within the intubation chamber 10 falls below a certain configurable level.

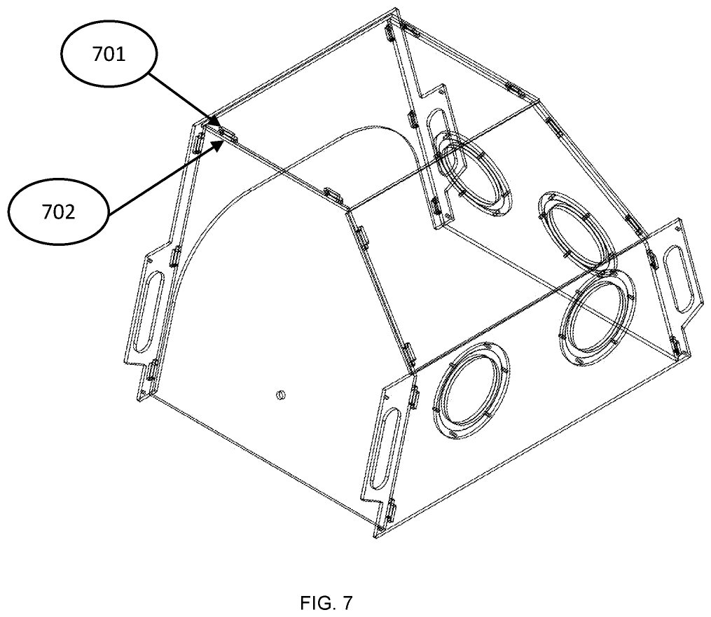

FIG. 7 is an example of a coupler used to connect various parts of the intubation chamber, according to some embodiments. As shown in FIG. 7 a hook slot coupler comprises hook 701 to couple with slot 702. The hook slot coupler makes shipping and assembly of the intubation chamber 10 easy as compared with using glues for coupling various members of intubation chamber 10. Assembly with the hook slot coupler along with bar handles 75 and 55 facilitates the assembly and structural integrity of intubation chamber 10.

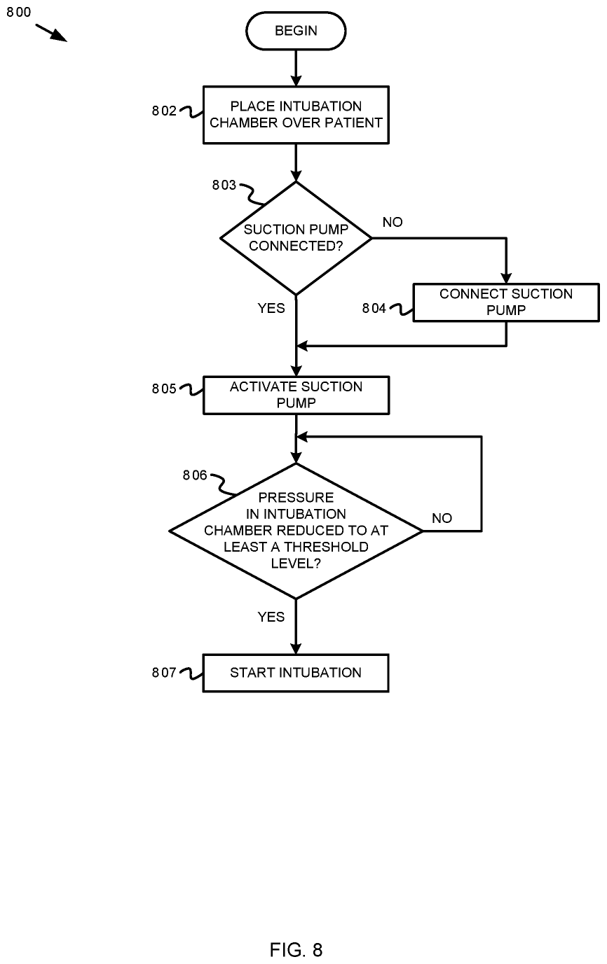

FIG. 8 is a flowchart of operations of using the intubation chamber, according to some embodiments. Operations of a flowchart 800 are described in reference to FIGS. 1 and 6. Operations of the flowchart 800 start at block 802.

At block 802, the intubation chamber 10 (having a transparent angled member 40 coupled to a vertical member 20, and an enclosure member 30) is placed over a patient. Operations of the flowchart 800 can include coupling a suction pump, not shown, to a nozzle 71 on the intubation chamber 10. The nozzle 71 can prevent the air from a suction pump to back flow into the intubation chamber 10.

At block 803, a determination is made of whether the suction pump is connected to the nozzle 71. If the suction pump is not connected, operations of the flowchart 800 continue at block 804. Otherwise, operations of the flowchart 800 continue at block 805.

At block 804, the suction pump is connected to nozzle 71. Operations of the flowchart 800 continue at block 805.

At block 805, the suction pump is activated.

At block 806, a determination is made of whether the pressure within the intubation chamber has been reduced to at least a threshold level. For example, the threshold level can be the atmospheric pressure outside the intubation chamber. In some embodiments, the threshold level can be a negative pressure such that any contaminants within the intubation chamber 10 removed the intubation chamber via the suction pump. This also ensures a vacuum seal between the intubation chamber, the patient, the users of the intubation chamber, and the table on which the patient is laying. In some embodiments, the inflatable seal referred to in FIG. 1 and FIG. 6 may be inflated to seal against the torso of the patient. If the pressure within the intubation chamber has not been reduced to at least the threshold level, operations of the flowchart 800 remain at block 805 to allow for continued pressure reduction. Otherwise, operations of the flowchart 800 continue at block 807.

At block 807, intubation is started. For example, intubation can be started by a user reaching through the vertical member 20 via one or more arm ports 21 to intubate the patient.

Although described with reference to example embodiments, those skilled in the art will recognize that changes may be made in form and detail. As such, it is intended that the foregoing detailed description be regarded as illustrative rather than limiting.

Use of the phrase “at least one of” preceding a list with the conjunction “and” should not be treated as an exclusive list and should not be construed as a list of categories with one item from each category, unless specifically stated otherwise. A clause that recites “at least one of A, B, and C” can be infringed with only one of the listed items, multiple of the listed items, and one or more of the items in the list and another item not listed.

What is claimed is:

1. An intubation chamber comprising:
a vertical member comprising at least one arm port;
an enclosure member comprising at least one access port; and
a transparent angled member coupled to the vertical member and the enclosure member, said at least one arm port to allow access to intubate a patient.

2. The intubation chamber of claim 1 wherein the vertical member and the enclosure member are transparent.

3. The intubation chamber of claim 1 further comprising at least one of sealable arm ports, sealable access ports, and a sealable opening that includes a curtain.

4. The intubation chamber of claim 1 further comprising a nozzle to connect a suction pump to maintain a negative pressure within the intubation chamber.

5. The intubation chamber of claim 4 further comprising an alarm to sound in response to the negative pressure falling below a predetermined level.

6. The intubation chamber of claim 1 wherein the transparent angled member comprises at least one of a convex lens and a Fresnel lens.

7. The intubation chamber of claim 1 wherein the transparent angled member is coupled to at least one of of the vertical member or the enclosure member using a hinge or a snap on coupler, a hook slot coupler, and a press fit.

8. The intubation chamber of claim 1 wherein the enclosure member further comprises a back member, an upper member, a left side wall and a right side wall, wherein each of the side walls are at an angle that is less than 90 degrees to a base of the intubation chamber.

9. The intubation chamber of claim 1 wherein at least one of the at least one arm port and the at least one access port is an iris port.

10. The intubation chamber of claim 1 wherein at least one of the vertical member, the enclosure member and the transparent angled member are comprised of at least one of polymethyl methacrylate, plastic, and polycarbonate.

11. The intubation chamber of claim 1 wherein the transparent angled member forms an angle Theta with an upper member of the enclosure member, wherein said angle Theta has a range of between 5 degrees and 85 degrees.

12. The intubation chamber of claim 1 wherein the enclosure member further comprises an opening with a seal to seal the intubation chamber against the torso of a patient.

13. The intubation chamber of claim 1 further comprises at least one sensor.

14. The intubation chamber of claim 13 wherein the sensor comprises a pressure sensor.

15. A method to intubate a patient comprising:
placing an intubation chamber, having a transparent angled member coupled to a vertical member, and an enclosure member, over a patient;
coupling a suction pump to a nozzle on the intubation chamber;
maintaining a negative pressure within the intubation chamber; and
reaching through the vertical member, via one or more arm ports, to intubate the patient.

16. The method of claim 15 wherein maintaining a negative pressure within the intubation chamber comprises removing air from the intubation chamber using the suction pump.

17. The method of claim 15 further comprising forming a vacuum seal between the intubation chamber, the patient and a surface on which the patient is laying.

18. The method of claim 17 wherein the vacuum seal is formed using elastomer seals.

19. The method of claim 15 wherein the intubation chamber is comprised of at least one of polymethyl methacrylate, polycarbonate, and plastic.

20. The method of claim 15 wherein reaching through the vertical member via one or more arm ports to intubate the patient comprises reaching through iris ports.
